SEQUENCE LISTING (6 sequences)

SEQ ID NO: 1 - DNA, 30 bp, Artificial Sequence
  synthetic E4 specific PCR Primer No. 1 for cloning Ad2 E4 sequence
aattgcagaaaatttaaattcatttttcat

SEQ ID NO: 2 - DNA, 29 bp, Artificial Sequence
  synthetic E4 specific PCR Primer No. 2 for cloning Ad-2 E4 sequence
gagtaacttgtatgttctagaattgtagt

SEQ ID NO: 3 - DNA, 21 bp, Artificial Sequence
  synthetic E4 specific PCR Primer No. 3 for cloning Ad-5 E4 sequence
actagtttcgcgccctttctc

SEQ ID NO: 4 - DNA, 17 bp, Artificial Sequence
  synthetic E4 specific PCR Primer No. 4 for cloning Ad-5 E4 sequence
ttaattaacatctacaa

SEQ ID NO: 5 - DNA, 31 bp, Artificial Sequence
  synthetic site-directed mutagenesis Primer No. 5 to create fragment mAd-5-E4
aattgcagaaaattttaaattcatttttcat

SEQ ID NO: 6 - DNA, 30 bp, Artificial Sequence
  synthetic site-directed mutagenesis Primer No. 6 to create fragment mAd-5-E4
actacaattctagacacatacaagttactc

(19) United States
(12) Patent Application Publication
(10) Pub. No.: US 2008/0166322 A1
(43) Pub. Date: Jul. 10, 2008

(54) METHODS FOR PRODUCING AN ADENOVIRUS TYPE 5 GENE TRANSFER VECTOR

(76) Inventors: Richard L. Atkinson, Richmond, VA (US); Suzanne E. Barbour, Richmond, VA (US); W. Palmer Wilkins, Richmond, VA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/953,306

(22) Filed: Dec. 10, 2007

Related U.S. Application Data
(60) Provisional application No. 60/869,152, filed on Dec. 8, 2006.

(51) Int. Cl. A01N 63/00; C12N 7/04; C12N 7/00; C12N 5/00

(52) U.S. Cl. 424/93.6; 435/236; 435/235.1; 435/325

(57) ABSTRACT

pZerotgCMV, an Ad-5-based expression vector in current use for gene transfer stimulates lipogenic enzymes within HepG2 cells (human hepatic carcinoma cells) and primary rat hepatocytes in vitro. Evidence indicates increased lipid accumulation in infected cells compared to uninfected cells. Therefore, inactivation of the E4 ORF1 gene or E4 gene cluster whether by replacement, removal, mutation, or use of antisense RNA encoded by the Ad-5 genome may prevent activation of lipogenic genes and subsequent lipid accumulation. Removal of just the E4 gene from pZerotgCMV may prevent both replication and stimulation of lipogenic enzymes.

### CROSS-REFERENCE TO RELATED APPLICATIONS

This application claims priority to and benefit under 35 U.S.C. § 119(e) to provisional application No. 60/869,152, filed Dec. 8, 2006, the disclosure of which is herein expressly incorporated by reference in its entirety.

### STATEMENT REGARDING FEDERALLY FUNDED RESEARCH

This invention was made, at least in part, with U.S. government support under NIH grant no: F3 INS051090 and grant no. NSF MCB-0544068. The U.S. government may have certain rights in the invention.

### FIELD OF THE INVENTION

The invention generally relates to an adenovirus vector that has been modified to prevent stimulation of lipogenic enzymes in cells and organs that are capable of synthesizing and storing triglycerides.

### BACKGROUND OF THE INVENTION

### Related Art

In humans, adenovirus infections cause acute upper respiratory tract infections, enteritis, or conjunctivitis. Some adenoviruses, such as adenovirus type 36, have been shown to be associated with obesity. (see, e.g., U.S. Pat. Nos. 6,127,113 and RE35,544). There are six major subgroups (A-F) that have been described for the human adenoviruses. Each subgroup has a number of specific serotypes. Adenoviruses are eukaryotic DNA viruses that can be modified to efficiently deliver a therapeutic or reporter transgene to a variety of cell types. Human adenoviruses are comprised of a linear, approximately 36 kb double-stranded DNA genome, which is divided into 100 map units (m.u.), each of which is 360 bp in length. The DNA contains short inverted terminal repeats (ITR) at each end of the genome that are required for viral DNA replication.

The genome of adenoviruses is complex and contains over 50 open reading frames (ORFs). These ORFs are overlapping and genes encoding one protein are often embedded within genes coding for other adenovirus proteins. The replicative cycle of adenovirus is divided into 2 phases; an early phase and a late phase. Early events include adsorption, penetration, transcription, and translation of an early set of genes. The early gene products mediate viral gene expression, DNA replication, induce cell cycle progression, and block apoptosis and include E1A, E1B, E2A, E2B, E3 and E4 genes. Once viral replication is initiated, the late phase begins with the expression of late genes and assembly of progeny virions.

The E1A protein is the first transcription unit to be expressed and is involved in transcriptional regulation. The E1B protein has the ability to interfere with normal cellular regulators such as blocking p53 induced growth inhibition and apoptosis. The E2 region is subdivided into E2A and E2B and provide the machinery for viral DNA replication. The E3 are not essential for viral growth in cell culture, however the E3 proteins protect cells from death mediated by cytotoxic T cells and death-inducing cytokines such as tumor necrosis factor (TFG). FAS ligand, and TNF-related apoptosis-inducing ligand. The E4 region includes seven open reading frames (ORFs). Some of the ORFs of the E4 gene encode proteins contribute to cell cycle regulation and other ORFs are involved in host cell transformation. The products of the late genes involves 5 gene clusters (e.g., L1-5) and primarily encode for virion structural proteins. Upon complete assembly of the virus, the host cell is ruptured and the virions are released for subsequent infections.

Adenovirus vectors have been exploited for the delivery of foreign genes to cells for a number of reasons. For example, adenoviral vectors have been shown to be highly effective for the transfer of genes into a wide variety of tissues in vivo and adenovirus have the capacity to infect both dividing and non-dividing cells; a number of tissues which are targets for gene therapy comprise largely non-dividing cells.

Adenovirus type 5 (subgroup C) is commonly used as vector for gene delivery. Adenoviruses can infect a broad range of mammalian cell types in vitro, but when injected intravenously, over 90% is concentrated in the liver. The fiber proteins on the adenovirus capsid are recognized by cell surface receptors, followed by receptor-mediated endocytosis. The adenovirus escapes the lysosomal compartment and enters the nucleus, where viral DNA replication takes place. Current Ad-5 vectors for gene transfer predominantly consist of altered viruses with the E1A gene of the virus removed.

One disadvantage of the current generation of adenovirus type 5 vectors is the capacity of the vector to alter lipid producing enzymes in the host cell as demonstrated herein, infra. The enhanced lipogenic enzymes in the target cells and organisms causes the accumulation of fat and increased insulin sensitivity, and ultimately may lead to increased body fat, obesity, and other associated complications.

### SUMMARY OF THE INVENTION

The invention provides a method for modifying or replacing the E4 gene along with the E1 gene of adenovirus type 5 vector to prevent both the replication of the virus and the alteration of lipid producing enzymes in the target cells and/or organism.

According to one aspect of the invention, a method of preventing an adenovirus type 5 vector from stimulating lipogenic enzymes in a subject requiring gene therapy is provided. The method may include inactivating an adenovirus type 5 E4 protein by modifying the adenovirus type 5 vector, such that E4 protein is incapable of stimulating lipogenic enzymes in the subject. Furthermore, the method may also include administering the modified adenovirus type 5 vector to the subject requiring gene therapy. The subject may be a human or an animal. The lipogenic enzymes may include sterol regulatory element binding protein and fatty acid synthesis.

The modification may include an insertion, a functional deletion, RNAi, and mutagenesis. In particular, the modification may include replacing the adenovirus type 5 E4 region with an adenovirus type 2 nucleic acid encoding an E4 gene product. More specifically, the adenovirus type 2 nucleic acid encoding an E4 gene product by an adenovirus type 2 nucleic acid encoding an E4 ORF1 gene product.

According to another aspect of the invention, a recombinant adenovirus type 5 vector having a modified E4 region where a nucleic acid encoding an adenovirus type 5 E4 protein has been replaced with a nucleic acid encoding an E4 protein of adenovirus type 2 is provided. The nucleic acid encoding the E4 protein of adenovirus type 2 may be a nucleic acid encoding an E4 ORF1 protein of adenovirus type 2. The recombinant adenovirus type 5 vector may further include a gene of interest. An isolated cell may include the modified adenovirus type 5 vector. The cell may be a mammalian cell. Moreover, a pharmaceutical composition may include the recombinant adenovirus type 5 vector and a suitable excipient.

According to a further aspect of the invention, a recombinant adenovirus type 5 vector may include a precursor RNAi molecule capable of inhibiting the expression of an adenovirus type 5 E4 specific messenger RNA (mRNA) to which it corresponds is provided. The RNAi molecule may include a first oligonucleoide strand having a length in a range of about 22 nucleotides to about 30 nucleotides, and a second oligonucleotide strand having a length in a range of about 22 nucleotides to about 20 nucleotides. The nucleotide sequence may be complementary to a sequence of an RNA of a target gene to direct target-specific RNAi.

The second oligonucleotide strand may anneal to the first oligonucleotide strand under biological conditions. The adenovirus type-5 specific mRNA may be an adenovirus type 5 E4 ORF1 mRNA. The recombinant adenovirus type 5 vector may further include a gene of interest. A cell may include the recombinant adenovirus type 5 vector having the RNAi precursor. The cell may be a mammalian cell.

Additional features, advantages, and embodiments of the invention may be set forth or apparent from consideration of the following detailed description, and claims. Moreover, it is to be understood that both the foregoing summary of the invention and the following detailed description are exemplary and intended to provide further explanation without limiting the scope of the invention as claimed.

### DESCRIPTION OF THE INVENTION

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It also is be noted that as used herein and in the appended claims, the singular forms “a,” “an,” and “the” include the plural reference unless the context clearly dictates otherwise. Thus, for example, a reference to “a virus” is a reference to one or more viruses and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law. Moreover, it is noted that like reference numerals reference similar parts throughout the several views of the drawings.

Moreover, provided immediately below is a “Definition” section, where certain terms related to the invention are defined specifically for clarity, but all of the definitions are consistent with how a skilled artisan would understand these terms. Particular methods, devices, and materials are described, although any methods and materials similar or equivalent to those described herein can be used in the practice or testing of the invention. All references referred to herein are incorporated by reference herein in their entirety.

### DEFINITIONS

“Nucleic acid sequences,” as the term is used herein, generally refers to nucleic acid sequences encoding a specific gene or functional equivalent thereof including those with deletions, insertions, or substitutions of different nucleotides resulting in a polynucleotide that encodes the same or a functionally equivalent or specific protein. Included within this definition are polymorphisms which may or may not be readily detectable using a particular oligonucleotide probe of the polynucleotide encoding a protein and improper or unexpected hybridization to alleles, with a locus other than the normal chromosomal locus for the polynucleotide sequence encoding a polypeptide. The encoded protein may also be “altered” and contain deletions, insertions, or substitutions of amino acid residues which produce a silent change and result in a functionally equivalent protein. Deliberate amino acid substitutions may be made on the basis of similarity in polarity, charge, solubility, hydrophobicity, hydrophilicity, and/or the amphipathic nature of the residues as long as the biological or immunological activity of a protein is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine, glycine and alanine, asparagine and glutamine, serine and threonine, and phenylalanine and tyrosine.

Nucleic acid molecules of the invention can be in the form of RNA, such as mRNA, hnRNA, tRNA, siRNA, hRNA, or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acids of the invention can include nucleic acid molecules comprising an open reading frame (ORF), but not limited to, at least one specified portion of an adenovirus such as the early phase genes (e.g., genes encoding the E1A, E1B, E2, E3, E4 proteins), intermediate genes (e.g., genes encoding the IVa2 and IX proteins), and the late phase genes (e.g., genes encoding L1, L2, L3, L4, and L5 proteins).

The invention also provides isolated nucleic acids that hybridize under selective hybridization conditions to a polynucleotide disclosed herein. Thus, the polynucleotides may be used for isolating, detecting, and/or quantifying nucleic acids comprising such polynucleotides. For example, the polynucleotides may be used to identify, isolate or amplify partial or full-length clones in a deposited library. Low or moderate stringency hybridization conditions are typically, but not exclusively, employed with sequences having a reduced sequence identity relative to complementary sequences. Moderate or high stringency conditions may be optionally employed for sequences of greater identity. Low stringency conditions allow selective hybridization of sequences having about 70% sequence identity and can be employed to identify orthologous or paralogous sequences.

The term “primer” generally refers to an oligonucleotide which is capable of acting as a point of initiation of synthesis when placed under conditions in which primer extension is initiated. An oligonucleotide “primer” may occur naturally, as in a purified restriction digest or may be produced synthetically.

A primer is selected to be “substantially” complementary to a strand of specific sequence of the template. A primer must be sufficiently complementary to hybridize with a template strand for primer elongation to occur. A primer sequence need not reflect the exact sequence of the template. For example, a non-complementary nucleotide fragment may be attached to the 5′ end of the primer, with the remainder of the primer sequence being substantially complementary to the strand. Non-complementary bases or longer sequences may be interspersed into the primer, provided that the primer sequence has sufficient complementarity with the sequence of the template to hybridize and thereby form a template primer complex for synthesis of the extension product of the primer.

The term “gene” refers to a DNA sequence that comprises control and coding sequences necessary for the production of a polypeptide or precursor thereof. The polypeptide can be encoded by a full length coding sequence or by any portion of the coding sequence so long as the desired enzymatic activity is retained. The term “gene” encompasses both cDNA and genomic forms of a given gene.

An “antisense gene,” as used herein, may be constructed by reversing the orientation of the gene with respect to its promoter so that the antisense strand is transcribed.

An “antisense RNA,” as used herein generally refers to an RNA molecule complementary to a particular RNA transcript that can hybridize to the transcript and block its function.

The terms “complementary” or “complementarity,” as used herein, include the natural binding of polynucleotides permissive conditions by base-pairing. For example, the sequence “A-G-T” binds to the complementary sequence “T-C-A.” Complementarity between two single-stranded molecules may be “partial,” in which only some of the nucleic acids bind, or it may be complete when total complementarity exists between the single stranded molecules. The degree of complementarity between nucleic acid strands has significant effects on the efficiency and strength of hybridization between nucleic acid strands. This is of particular importance in antisense or nonsense nucleic acid reactions, such as antisense RNA, which depend upon binding between nucleic acids strands and in the design and use of molecules.

The term “deletion,” as used herein, generally refers to a change in the amino acid or nucleotide sequence and results in the absence of one or more amino acid residues or nucleotides.

The term “insertion” or “addition,” as used herein, generally includes a change in an amino acid or nucleotide sequence resulting in the addition of one or more amino acid residues or nucleotides, respectively, as compared to the naturally occurring molecule.

The term “introduction,” as used herein generally refers to insertion of a nucleic acid sequence into a cell, by methods including infection, transfection, transformation or transduction.

An “expression cassette,” as used herein generally refers to a combination of regulatory elements that are required by the host for the correct transcription and translation (expression) of the genetic information contained in the expression cassette. These regulatory elements comprise a suitable (i.e., functional in the selected host) transcription promoter and a suitable transcription termination sequence.

“Promoter,” as used herein generally includes a regulatory region of DNA capable of initiating, directing and mediating the transcription of a nucleic acid sequence. Promoters may additionally include recognition sequences, such as upstream or downstream promoter elements, which may influence the transcription rate.

“Inducible promoter,” as used herein generally refers to a promoter where the rate of RNA polymerase binding and initiation is modulated by external stimuli. Such stimuli include light, heat, anaerobic stress, alteration in nutrient conditions, presence or absence of a metabolite, presence of a ligand, microbial attack, wounding and the like.

“Viral promoter,” as used herein generally refers to a promoter with a DNA sequence substantially similar to the promoter found at the 5′ end of a viral gene. A typical viral promoter is found at the 5′ end of the gene coding for the p2I protein of MMTV.

“Synthetic promoter,” as used herein generally refers to a promoter that was chemically synthesized rather than biologically derived. Usually synthetic promoters incorporate sequence changes that optimize the efficiency of RNA polymerase initiation.

“Constitutive promoter,” as used herein generally refers to a promoter where the rate of RNA polymerase binding and initiation is approximately constant and relatively independent of external stimuli.

“Inhibition,” as used herein, generally refers to a reduction in the parameter being measured. The amount of such reduction is measured relative to a standard (control). The preferred detection products may include newly transcribed mRNA and a DNA-DNABP complex. “Reduction” is defined herein as a decrease of at least about 25% relative to control, specifically at least about 50%, and specifically at least about 75%.

“Transfection,” as used herein includes the process of introducing a DNA expression vector into a cell. Various methods of transfection are possible including microinjection or lipofection. Transformation refers to a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the type of host cell being transformed and may include, but is not limited to, viral infection, electroporation, heat shock, and lipofection.

“Functional equivalent,” as used herein generally refers to a protein or nucleic acid molecule that possesses functional or structural characteristics that is substantially similar to a protein, polypeptide, enzyme, or nucleic acid. A functional equivalent of a protein may contain modifications depending on the necessity of such modifications for the performance of a specific function. The term “functional equivalent” is intended to include the “fragments,” “mutants,” “hybrids,” “variants,” “analogs,” or “chemical derivatives” of a molecule.

The term “purification,” as used herein, generally refers to any process by which proteins, polypeptides, or nucleic acids are separated from other elements or compounds on the basis of charge, molecular size, or binding affinity.

The phrase “substantially purified,” or “substantially isolated,” as used herein generally includes nucleic or amino acid sequences that are removed from their natural environment, isolated or separated, and are at least about 60% free, specifically at least about 75% free, and most specifically at least about 90% free from other components with which they may be associated with, and includes recombinant or cloned nucleic acid isolates and chemically synthesized analogs or analogs biologically synthesized by systems.

“Vector,” as used herein generally refers to a cloning vector that is designed so that a coding nucleic acid sequence inserted at a particular site will be transcribed and translated. A typical expression vector may contain a promoter, selection marker, nucleic acids encoding signal sequences, and regulatory sequences, e.g., polyadenylation sites, 5′-untranslated regions, and 3′-untranslated regions, termination sites, and enhancers. “Vectors” may include viral derived vectors, bacterial derived vectors, plant derived vectors, and insect derived vectors. Exemplary adenoviral vectors include adenovirus type 5 vectors such as pZerotgCMV.

The phrase “adenovirus gene coding region” refers to a nucleotide sequence containing more than one adenovirus gene coding gene. A “helper adenovirus” or “helper virus” refers to an adenovirus that is replication-competent in a particular host cell (the host may provide adenovirus gene products such as E1 proteins), this replication-competent virus may be used to supply in trans functions (e.g., proteins) which are lacking in a second replication-incompetent virus; the first replication-competent virus is said to “help” the second replication-incompetent virus thereby permitting the propagation of the second viral genome in the cell containing the helper and second viruses.

The term “lipogenic,” as used herein, generally refers to the ability to stimulate or cause the accumulation of fat in a target cell, tissue, and/or organ. Lipogenic enzymes include, without limitation, sterol regulatory element binding protein (SREBP-1), fatty acid synthetase (FAS), acetyl CoA carboxylase, and β-ketothiolase, and the like.

“Mammal,” as used herein, includes animals and humans. Thus, when referring to processes such as harvesting tissue from an animal, it is intended that the animal can be a human. Although at times reference may be made herein to “an animal or human,” that is not intended to imply that the term “animal” does not include a human.

“Subject,” as used herein, includes individuals who require intervention or manipulation due to a disease state, treatment regimen or experimental design. Furthermore, the term “subject” includes animals and humans.

The invention relates generally to methods and products for use in the field of gene therapy and gene transfer. In particular, the invention provides a modified adenovirus type vector. Specifically, the E4 gene of gene product of human adenovirus 5 vector may be either modified, removed, or inhibited to prevent stimulation of lipogenic enzymes in cells and organs that are capable of synthesizing and storing triglycerides. Alternatively, the adenovirus type 5 vector may be modified to include further nucleic acid sequences that encode for nucleic acid sequences or proteins that have the capacity to attenuate the E4 gene product; or moreover, the adenovirus type 5 vector may be co-transfected with a helper virus containing nucleic acid sequences encoding nucleic acid sequences or proteins that have the capacity to attenuate the E4 protein or prevent expression of the E4 protein.

Ad-36 belongs to subgroup D and is distinct in both neutralization and hemagglutination-inhibition from most other human adenoviruses. Specifically, it has been demonstrated that human Ad-36 stimulates the storage of fat and formation of new fat cells in mouse preadipocytes (3T3-L1 cells) and in human preadipocytes. Additionally, it has been demonstrated in U.S. Pat. Nos. 6,127,113 and 6,664,050 (now RE35,544), which are herein incorporated by reference in their entirety, that Ad-36 infection is associated with obesity. For example, four experiments were conducted in chickens, one experiment in mice, and two experiments in monkeys, all showing that infection with Ad-36 increased body fat and lowered serum cholesterol and triglycerides. Most notable, food intake measured in chickens, mice, and rats was not different between infected and control animals indicating that energy expenditure was different. The mechanism of Ad-36 has a direct effect on adipocytes to increase lipogenic enzymes and differentiation factors. Cells infected with Ad-36 exhibited about 2 times as much stored triglyceride at 5 days.

Specifically, it is the E4 gene of human adenovirus type 36 which stimulates lipogenic enzymes in adipocytes, including sterol regulatory element binding protein (SREBP-1) and fatty acid synthase (FAS), and is associated with increased number of adipocytes and increased fat accumulation within adipocytes in vitro and in vivo. However, until the invention described herein (as described below), no one has demonstrated that adenovirus type 5, which after intravenous injection concentrates primarily in the liver, affects these enzymes in the liver. The E4 gene is associated with stimulation of fat producing enzymes in multiple tissues, as described below, and use of adenovirus type 5 vectors that still contain the E4 gene will cause alteration of the cellular milieu with accumulation of fat within cells. Any experiment in which hepatic enzymes, intracellular fat content, body composition, or body weight are critical may be compromised by the Ad-5 vector currently on the market. Accordingly, one embodiment of the invention is directed to producing an Ad-5 vector that will not cause disease secondary to viral infection.

Human hepatoma cells (HepG2) and primary rat hepatocytes were infected with Ad-5 vector and screened for expression of Sterol Regulatory Element Binding Protein-1 (SREBP1), a transcription factor that induces expression of several lipogenic genes. Ad-5 induced SREBP1 expression in a dose dependent manner in both cell types. SREBP transcriptional activity was assayed with the pSynSRE reporter construct. Immunoblot and real time PCR analyses indicated that SREBP1 targets, acetyl CoA carboxylase (ACC) and fatty acid synthase (FAS) were induced in both hepatic cell types. Also, expression of SREBP1 was elevated in livers of mice infected with Ad-5. Increased fatty acid synthesis and lipid accumulation was shown by [¹⁴C]-acetate incorporation and Oil Red 0 staining, respectively. Thus, the Ad-5 vector promotes alteration of a number of enzymes and produces lipid accumulation in hepatocytes.

pZerotgCMV, an Ad-5-based expression vector in current use for gene transfer stimulated lipogenic enzymes within HepG2 cells (human hepatic carcinoma cells) and primary rat hepatocytes in vitro. Increased lipid accumulation in infected cells compared to uninfected cells was shown. Therefore, inactivation of the E4 gene whether by removal, mutation, or use of antisense RNA encoded by the Ad-5 genome may prevent activation of lipogenic genes and subsequent lipid accumulation. Removal of just the E4 gene from pZerotgCMV may prevent both replication and stimulation of lipogenic enzymes. Also, including a nucleic acid sequence within the Ad-5 genome that codes for anti-sense RNA will block translation of the E4 protein.

In one embodiment of the invention, the Ad-5 E4 gene sequence may be replaced with the E4 gene sequence from an adenovirus, which does not stimulate lipogenic enzymes and cause fat accumulation in the target cells. Specifically, the adenovirus type 2 E4 gene sequence may be used. A chimera adenoviral vector having primarily Ad-5 sequences, but have the E4 gene region replaced with Ad-2 sequences. Specifically, the Ad-5 E4 region may be substituted with any one of Ad-2 ORFs 1-7, and particularly, Ad-2 ORF1.

In another embodiment of the invention, the Ad-5 E4 gene may be modified by introducing deletions, insertions, or mutations of nucleic acid sequences into the E4 gene to rendered the E4 gene products incapable of activating lipogenic enzymes in the target cell. Method for the generating deletions, insertions and mutations is well known to those of skill in the art.

In a particular embodiment, the invention provides a precursor RNAi molecule capable of mediating the expression of a adenovirus type 5 virus-specific mRNA to which it corresponds having a first oligonucleotide strand that has a length in a range of about 15 nucleotides to about 30 nucleotides; and a second oligonucleotide strand that has a length of about 15 nucleotides to about 30 nucleotides and has a nucleotide sequence that is sufficiently complementary to a sequence of an RNA of the target gene to direct target-specific RNAi. The second oligonucleotide strand anneals to the first oligonucleotide strand under biological conditions. The RNAi molecule mediates RNA interference of a adenovirus-specific mRNA to which it corresponds. In one embodiment, the precursor RNAi molecule targets the gene that encodes E4 proteins of adenovirus type 5.

The invention also provides a precursor RNAi molecule capable of mediating the expression of a mRNA for a gene product that has the capacity to stimulate the lipogenic enzymes in a target cell to which it corresponds having a first oligonucleotide strand that has a length of about 15 nucleotides to about 30 nucleotides and a second oligonucleotide strand that has a length in a range of about 15 nucleotides to about 30 nucleotides. Moreover, the precursor RNAi molecule has a nucleotide sequence that is sufficiently complementary to a sequence of an RNA of the target gene to direct target-specific RNAi. The second oligonucleotide strand anneals to the first oligonucleotide strand under biological conditions and the RNAi molecule mediates RNA interference of the mRNA for the gene product that contributes to the stimulation of lipogenic enzyme in a target cell. In particular, the RNAi molecule of the invention has the capacity to inactivate the adenovirus type 5 E4 gene product, preventing stimulation of the lipogenic enzymes and ultimately the accumulation of fat in the target cells.

The invention also provides an expression cassette containing a nucleic acid encoding at least one strand of the precursor RNAi molecule described above. The expression cassette may further contain a promoter, such as inducible promote, a viral promoter, or a constitutive promoter. In certain embodiments the promoter may be a cytomegalovirus (CMV), rous sarcoma virus (RSV), pol II, or pol III promoter. In certain embodiments, the expression cassette may further contain a polyadenylation signal, such as a synthetic minimal polyadenylation signal. In certain embodiments, the expression cassette further contains a marker gene. The invention also provides a cell containing the expression cassette. The cell may be a mammalian cell. The invention further provides a non-human mammal containing the expression cassette.

The invention also provides a vector containing the expression cassette described above. The vector, in some embodiments, may contain two expression cassettes: a first expression cassette having a nucleic acid encoding the first strand of the RNA duplex, and a second expression cassette having a nucleic acid encoding the second strand of the RNA duplex. The invention further provides a vector containing an expression cassette having (1) a nucleic acid sequence encoding a first portion of RNA, (2) a second portion of RNA located immediately 3′ of the first portion of RNA, and (3) a third portion of RNA located immediately 3′ of the second portion of RNA, where the first and third portions of RNA are each less than about 30 nucleotides in length and each more than about 15 nucleotides in length. The sequence of the third portion of RNA may be the complement of the sequence of the first portion of RNA to form an RNA duplex. The RNA duplex may mediate RNA interference of a adenovirus type 5 mRNA to which it corresponds. The vectors described above may further contain a polyadenylation signal and/or a marker gene. The vector may be an adenoviral, lentiviral, adeno-associated viral (AAV), poliovirus, HSV, or murine Moloney-based viral vector. Alternatively, the vector may be a plasmid vector. The invention provides a composition of a polymer or excipient and the vector.

The adenovirus type 5 vectors according to the invention may be used to deliver genes or nucleic acids of interest to host cells. Compositions suitable for such a use are also part of the invention. The amount of adenovirus type 5 vector that needs to be present per dose or per infection (“m.o.i.”) will depend on the condition to be treated, the route of administration (typically parenteral), the subject, and the efficiency of infection, etc. Dose finding studies are well known in the art and those already performed with other (adenoviral) gene delivery vehicles may be used as guides to find suitable doses of the adenovirus type 5 vectors according to the invention. In general, one skilled in the art may also find suitable excipients, suitable means of administration, suitable means of preventing infection with the adenovirus type 5 vector where it is not desired, etc. Thus, the invention also provides a pharmaceutical formulation comprising a adenovirus type 5 vector according to the invention and a suitable excipient, as well as a pharmaceutical formulation comprising an adenovirus, a chimera thereof, or a functional homologue thereof according to the invention and a suitable excipient.

### Construction of Nucleic Acids

The isolated nucleic acids of the invention can be made using (a) recombinant methods, (b) synthetic techniques, (c) purification techniques, or combinations thereof, as well-known in the art.

The nucleic acids may include sequences in addition to a polynucleotide of the invention. For example, a multi-cloning site comprising one or more endonuclease restriction sites can be inserted into the nucleic acid to aid in isolation of the polynucleotide. Also, translatable sequences may be inserted to aid in the isolation of the translated polynucleotide of the invention. For example, a hexa-histidine marker sequence provides a convenient means to purify the proteins of the invention. The nucleic acids of the invention—excluding the coding sequence—may be optionally a vector, adapter, or linker for cloning and/or expression of a polynucleotide of the invention.

Additional sequences may be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art.

### Recombinant Methods for Constructing Nucleic Acids

The isolated nucleic acid compositions of this invention, such as RNA, cDNA, genomic DNA, or any combination thereof, can be obtained from biological sources using any number of cloning methodologies known to those of skill in the art. In some embodiments, oligonucleotide probes that selectively hybridize, under stringent conditions, to the polynucleotides of the invention may be used to identify the desired sequence in a cDNA or genomic DNA library. The isolation of RNA, and construction of cDNA and genomic libraries, is well known to those of ordinary skill in the art.

### Nucleic Acid Screening and Isolation Methods

A cDNA or genomic library can be screened using a probe based upon the sequence of a polynucleotide of the invention, such as those disclosed herein. Probes can be used to hybridize with genomic DNA or cDNA sequences to isolate homologous genes in the same or different organisms. Those of ordinary skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent such as formamide. For example, the stringency of hybridization is conveniently varied by changing the polarity of the reactant solution through, for example, manipulation of the concentration of formamide within the range of about 0% to about 50%. The degree of complementarity (sequence identity) required for detectable binding will vary in accordance with the stringency of the hybridization medium and/or wash medium. The degree of complementarity may be about 100%, or about 70% to about 100%, or any range or value therein. However, it should be understood that minor sequence variations in the probes and primers may be compensated for by reducing the stringency of the hybridization and/or wash medium.

Methods of amplification of RNA or DNA are well known in the art and can be used according to the invention without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; U.S. Pat. No. 4,795,699 and U.S. Pat. No. 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the disclosures of all references in this paragraph are incorporated by reference herein in their entirety.

For example, polymerase chain reaction (PCR) technology may be used to amplify the sequences of polynucleotides of the invention and related genes directly from genomic DNA or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

### Synthetic Methods for Constructing Nucleic Acids

The isolated nucleic acids of the invention may also be prepared by direct chemical synthesis by known methods. Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Recombinant Expression Cassettes

The invention further provides recombinant expression cassettes having a nucleic acid of the invention. A nucleic acid sequence of the invention, for example a cDNA or a genomic sequence encoding an adenovirus protein of the invention, such as the E4 ORF-1 sequence of adenovirus type 2, may be used to construct a recombinant expression cassette that can be introduced into at least one desired host cell. A recombinant expression cassette will typically comprise a polynucleotide of the invention operably linked to transcriptional initiation regulatory sequences that will direct the transcription of the polynucleotide in the intended host cell. Both heterologous and non-heterologous (i.e., endogenous) promoters can be employed to direct expression of the nucleic acids of the invention.

In some embodiments, isolated nucleic acids that serve as promoter, enhancer, or other elements may be introduced in the appropriate position (upstream, downstream or in intron) of a non-heterologous form of a polynucleotide of the invention so as to up or down regulate expression of a polynucleotide of the invention. For example, endogenous promoters can be altered in vivo or in vitro by mutation, deletion and/or substitution.

### Vectors

The invention also relates to vectors that include isolated nucleic acid molecules of the invention, host cells that are genetically engineered with the recombinant vectors, and the production of at least one adenovirus protein such as the Ad-5 E4 protein, by recombinant techniques, as is well known in the art.

The polynucleotides may optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs may include a translation initiating at the beginning and a termination codon (e.g., UAA, UGA or UAG) appropriately positioned at the end of the mRNA to be translated.

Expression vectors may include at least one selectable marker. Such markers include, e.g., but not limited to, methotrexate (MTX), dihydrofolate reductase (DHFR, U.S. Pat. Nos. 4,399,216; 4,634,665; 4,656,134; 4,956,288; 5,149,636; 5,179,017, colormetric markers such as GFP and beta-galactosidase, ampicillin, neomycin (G418), mycophenolic acid, or glutamine synthetase (U.S. Pat. Nos. 5,122,464; 5,770,359; 5,827,739) resistance for eukaryotic cell culture, and tetracycline or ampicillin resistance genes for culturing in E. coli and other bacteria or prokaryotics. Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods.

Those of ordinary skill in the art are knowledgeable in the numerous expression systems available for expression of a nucleic acid encoding a protein of the invention, and thus there is no need to describe them herein.

The detailed description, definitions and examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled molecular biology or in the relevant fields are intended to be within the scope of the appended claims.

The disclosures of all references and publications cited above are expressly incorporated by reference in their entireties to the same extent as if each were incorporated by reference individually.

### EXAMPLES

### Specific Example 1

### Construction of Ad-5/Ad-2 Chimera Vector

Construction of pAd-2_{E4}

The genome sequences for adenovirus type 5 and adenovirus type 2 may easily be accessed by those of ordinary skill in the art by their Genbank accessions numbers BK000408 and BK000407, respectively.

Ad-5/Ad-2 chimera vector is an adenovirus 5 based vector with an Ad-2 E4 substitution. The 3,000 bp DNA fragment encoding E4 (i.e., spanning nucleotides 32,832 to 35,798 of Ad-2) is derived by polymerase chain reaction of Ad2 DNA with E4 specific DNA primers:

| | |
|---|---|
| Primer No. 1: | |
| AATTGCAGAA AATTTAAATT CATTTTTCAT | (SEQ ID No. 1) |
| Primer No. 2: | |
| GAGTAACTTG TATGTTCTA GAATTGTAGT | (SEQ ID No. 2) |

Additional sequences supplied by the oligonucleotides include a cloning site at the 5′ and 3′ ends of the PCR fragment (SwaI and XbaI, respectively). The PCR fragment is first ligated into a ADEasy-1 vector linearized with SwaI and XbaI to create plasmid Ad-2_{E4}. Plasmid Ad-2_{E4} is sequenced for verification of successful cloning of Ad-2 E4 sequence.

Construction of pAd-5_{E4}

A 6,222 bp DNA fragment encoding a region of Ad-5 including E4 is derived by polymerase chain reaction of Ad5 DNA with E4 specific DNA primers:

| | |
|---|---|
| Primer No. 3: | |
| ACTAGTT TCGCGCCCTT TCTC | (SEQ ID No. 3) |
| Primer No. 4: | |
| TTAATT AACATCTACAA | (SEQ ID No. 4) |

Additional sequences supplied by the oligonucleotides include a cloning site at the 5′ and 3′ ends of the PCR fragment (BamHI and NotI, respectively). The PCR fragment is first ligated into a ADEasy-1 vector linearized with BamHI and NotI to create plasmid pAd-5 _{E4}. Plasmid pAd-5 _{E4} is sequenced for verification of successful cloning of Ad-2 E4 sequence.

pAd-5 _{E4} is subjected to site-directed mutagenesis to generate XbaI and SwaI restriction enzymes sites in the 6,222 base pair insert of pAd-5 _{E4} to create fragment mAd-5_{E4} using the primers:

| | |
|---|---|
| Primer No. 5: | |
| AATTGCAGAA AATTTTAAATT CATTTTTCAT | (SEQ ID No. 5) |
| Primer No. 6: | |
| ACTACAATTC TAGACACATA CAAGTTACTC | (SEQ ID No. 6) |

Fragment mAd-5_{E4} is digested with PacI and SpeI and cloned into a PADEasy vector linearized with SpeI and PacI using standard cloning techniques known to those of skill in the art to create plasmid pmAd-5_{E4}.

Plasmid pAd-2_{E4} is digested with SwaI and XbaI to liberate the 3,000 bp Ad-2 E4 fragment. This fragment is cloned into a pmAd-5_{E4} vector linearized with the XbaI and SwaI restriction enzymes to create plasmid pAd-5_{AD2E4}. The fragment is validated by DNA sequencing.

### Construction of Ad5/Ad2 Chimeric Adenovirus

To generate the recombinant Ad5/Ad2 adenovirus, the plasmid pAd-5_{AD2E4} is digested with XbaI and SwaI. The 6,222 base pair fragment is ligated to Ad-5 DNA digested with XbaI and SwaI. Following ligation, the reaction is used to transfect the appropriate host cells by the calcium phosphate procedure. About 7-8 days following transfection, a single plaque appears and is used to reinfect a dish of appropriate host cells. Total DNA is prepared from the infected cells and analyzed by restriction analysis with multiple enzymes to verify the integrity of the construct. Viral supernatant is then used to infect appropriate host cells and are validated for the presence of Ad-2 E4 protein using an immunoprecipitation assay. Following these verification procedures, the virus is further purified by two rounds of plaque purification.

Plaque purified virus is grown into a small seed stock by inoculation at low multiplicities of infection onto appropriate cells grown in monolayers in appropriate medium supplemented with 10% bovine calf serum.

1. A method of preventing an adenovirus type 5 vector from stimulating lipogenic enzymes in a subject requiring gene therapy, said method comprising the step of:
inactivating an adenovirus type 5 E4 protein by modifying the adenovirus type 5 vector, such that E4 protein is incapable of stimulating lipogenic enzymes in the subject.

2. The method of claim 1, further comprising the step of administering the modified adenovirus type 5 vector to the subject requiring gene therapy.

3. The method of claim 1, wherein the modification in said inactivating step is one or more selected from the group consisting of an insertion, a functional deletion, RNAi, and mutagenesis.

4. The method of claim 1, wherein the modification in said inactivating step comprises replacing the adenovirus type 5 E4 region with an adenovirus type 2 nucleic acid encoding an E4 gene product.

5. The method of claim 4, wherein the nucleic acid encoding the E4 protein is an adenovirus type 2 nucleic acid encoding an E4 ORF1 gene product.

6. The method of claim 1, wherein the lipogenic enzymes are selected from the group consisting of sterol regulatory element binding protein and fatty acid synthesis.

7. The method of claim 1, wherein the subject is a human.

8. The method of claim 1, wherein the subject is an animal.

9. A recombinant adenovirus type 5 vector comprising a modified E4 region where a nucleic acid encoding an adenovirus type 5 E4 protein has been replaced with a nucleic acid encoding an E4 protein of adenovirus type 2.

10. The recombinant adenovirus type 5 vector of claim 9, wherein the nucleic acid encoding the E4 protein of adenovirus type 2 is a nucleic acid encoding an E4 ORF1 protein of adenovirus type 2.

11. The recombinant adenovirus type 5 vector of claim 9, further comprising a gene of interest.

12. An isolated cell comprising an recombinant adenovirus type 5 vector of claim 9.

13. The isolated cell of claim 12, wherein the cell is a mammalian cell.

14. A pharmaceutical formulation comprising a recombinant adenovirus vector of claim 9 and a suitable excipient.

15. A recombinant adenovirus type 5 vector comprising:
a precursor RNAi molecule capable of inhibiting the expression of an adenovirus type 5 E4 specific messenger RNA (mRNA) to which it corresponds wherein the RNAi molecule includes a first oligonucleoide strand having a length in a range of about 22 nucleotides to about 30 nucleotides, a second oligonucleotide strand having a length in a range of about 22 nucleotides to about 20 nucleotides, and wherein the nucleotide sequence is complementary to a sequence of an RNA of a target gene to direct target-specific RNAi.

16. The recombinant adenovirus type 5 vector of claim 15, wherein said second oligonucleotide strand anneals to said first oligonucleotide strand under biological conditions.

17. The recombinant adenovirus type 5 vector of claim 15, wherein the adenovirus type-5 specific mRNA is an adenovirus type 5 E4 ORF1 mRNA.

18. The recombinant adenovirus type 5 vector of claim 15, further comprising a gene of interest.

19. An isolated cell comprising an recombinant adenovirus type 5 vector of claim 15.

20. The isolated cell of claim 19, wherein the cell is a mammalian cell.

21. A pharmaceutical formulation comprising a recombinant adenovirus vector of claim 19 and a suitable excipient.
